(19) United States
(12) Patent Application Publication
(10) Pub. No.: US 2026/0033796 A1
(43) Pub. Date: Feb. 5, 2026

(54) RADIOGRAPHIC IMAGING CONTROL APPARATUS, RADIOGRAPHIC IMAGING CONTROL METHOD, AND STORAGE MEDIUM

(71) Applicant: Konica Minolta, Inc., Tokyo (JP)

(72) Inventors: Kentaro HARA, Tokyo (JP); Takeshi NUKANOBU, Tokyo (JP)

(21) Appl. No.: 19/280,959

(22) Filed: Jul. 25, 2025

(30) Foreign Application Priority Data
Jul. 30, 2024 (JP) 2024-123025

(51) Int. Cl. A61B 6/00; A61B 6/46

(52) U.S. Cl. CPC A61B 6/54; A61B 6/461; A61B 6/563; A61B 6/56

(57) ABSTRACT

A radiographic imaging control apparatus includes a hardware processor and outputs a display-purpose image generated based on a series of frames, the series of frames being captured by moving image capturing of a subject with radiation. When the moving image capturing is intermittently performed multiple times from one time of imaging start to one time of imaging end, the hardware processor prioritizes displaying a display-purpose image of second moving image capturing over displaying a display-purpose image of first moving image capturing The second moving image capturing is performed later than the first moving image capturing.

### CROSS-REFERENCE TO RELATED APPLICATIONS

The entire disclosure of Japanese Patent Application No. 2024-123025 filed on Jul. 30, 2024, is incorporated herein by reference in its entirety.

### TECHNICAL FIELD

The present invention relates to a radiographic imaging control apparatus, a radiographic imaging control method, and a storage medium.

### DESCRIPTION OF RELATED ART

Intermittent capturing of moving images has been proposed to obtain images at necessary timing in, for example, an examination for checking the state of catheter insertion or the state in a subject immediately after administration of a contrast agent. However, when the wireless communication speed between a radiographic imaging apparatus and a display device is low, the frame obtaining speed by the display device may be lower than the designated frame rate. Since the display device delays in displaying the obtained frame images in the captured order, the frames may not be displayed at necessary timings.

Japanese Unexamined Patent Publication No. 2017-113344 proposes a method of preventing a delay in wirelessly transmitting captured frames. According to JP2017-113344A, a dynamic imaging apparatus transmits a first frame image that includes a region of interest of a subject and transmits a feature amount related to the dynamic state of the subject in second and subsequent frame images.

According to the known technology, it is possible to stably transmit frames obtained by moving image capturing. However, when imaging is performed multiple times in one session of moving image capturing, frames captured in current imaging may not be transmitted to the display device within the period of the current imaging, depending on the number of frames, image conditions, or the like. Frames that could not be transmitted during the current imaging are transmitted to the display device at the next imaging, so that the frames of the previous imaging are displayed at the next imaging. Therefore, in the respective times of imaging, frame images may not be displayed at necessary timing.

To solve the above-described problem, an object of the present invention is to provide a radiographic imaging control apparatus, a radiographic imaging control method, and a program capable of giving priority to displaying images of current intermittent imaging over displaying images of past intermittent imaging when intermittent imaging is newly started.

### SUMMARY OF THE INVENTION

To achieve at least one of the abovementioned objects, according to an aspect of the present invention, there is provided a radiographic imaging control apparatus that includes a hardware processor and outputs a display-purpose image generated based on a series of frames, the series of frames being captured by moving image capturing of a subject with radiation, wherein when the moving image capturing is intermittently performed multiple times from one time of imaging start to one time of imaging end, the hardware processor prioritizes displaying a display-purpose image of second moving image capturing over displaying a display-purpose image of first moving image capturing, the second moving image capturing being performed later than the first moving image capturing.

According to another aspect of the present invention, there is provided a radiographic imaging control method for a radiographic imaging control apparatus that outputs a display-purpose image generated based on a series of frames, the series of frames being captured by moving image capturing of a subject with radiation, wherein when the moving image capturing is intermittently performed multiple times from one time of imaging start to one time of imaging end, displaying a display-purpose image of second moving image capturing is prioritized over displaying a display-purpose image of first moving image capturing, the second moving image capturing being performed later than the first moving image capturing.

According to another aspect of the present invention, there is provided a non-transitory computer-readable storage medium storing a program that causes a computer of a radiographic imaging control apparatus that outputs a display-purpose image generated based on a series of frames, the series of frames being captured by moving image capturing of a subject with radiation, wherein when the moving image capturing is intermittently performed multiple times from one time of imaging start to one time of imaging end, the program causes the computer to prioritize displaying a display-purpose image of second moving image capturing over displaying a display-purpose image of first moving image capturing, the second moving image capturing being performed later than the first moving image capturing.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 illustrates an example of the entire configuration of an in-hospital system including a dynamic imaging system according to the first embodiment;

FIG. 2 is a block diagram of a main body according to the first embodiment;

FIG. 3 schematically illustrates pulse irradiation in a normal mode in a known art;

FIG. 4 schematically illustrates pulse irradiation in an intermittent imaging mode according to the first embodiment;

FIG. 5 is a flowchart of an operation example of a controller of a main body in executing the intermittent imaging mode according to the first embodiment;

FIG. 6 illustrates an example of an imaging screen displayed on a display part according to the first embodiment;

FIG. 7 illustrates a flow of intermittent imaging by the FPD, frame acquisition by the controller of the main body, and image display by the display part of the main body when the intermittent imaging mode is executed according to the first embodiment;

FIG. 8 illustrates a flow of intermittent imaging by the FPD, frame acquisition by the controller of the main body, and image display by the display part of the main body when the intermittent imaging mode is executed according to a first modification example;

FIG. 9 illustrates a flow of intermittent imaging by the FPD, frame acquisition by the controller of the main body, and image display by the display part of the main body when the intermittent imaging mode is executed according to a second modification example;

FIG. 10 illustrates an example of various kinds of information displayed on the imaging screen of the display part before the intermittent imaging mode is executed according to the second embodiment; and

FIG. 11 illustrates an example of various kinds of information displayed on the imaging screen of the display part while the intermittent imaging mode is executed according to the second embodiment.

### DETAILED DESCRIPTION

A radiographic imaging control apparatus, a radiographic imaging control method, and a program according to preferred embodiments of the present disclosure will be described in detail below with reference to the accompanying drawings. However, the scope of the invention is not limited to the disclosed embodiments.

### First Embodiment

### Configuration Example of Dynamic Imaging System 10

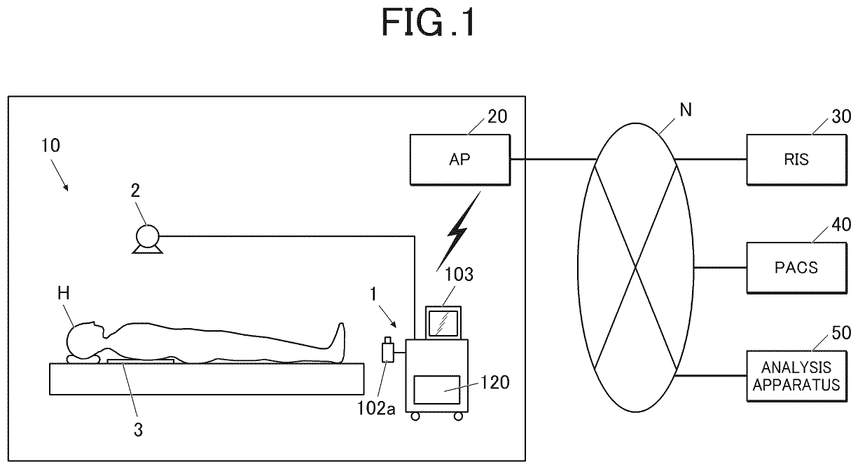

FIG. 1 illustrates an example of the entire configuration of an in-hospital system including a dynamic imaging system 10 according to the first embodiment. The dynamic imaging system 10 is, for example, a system for performing dynamic imaging in a round visit for patients who are difficult to move. The dynamic imaging system 10 includes a main body 1, a radiation source 2, and an FPD 3.

The main body 1 has wheels and is configured as a movable medical cart. Note that the dynamic imaging system 10 may be portable without wheels. The main body 1 is connected to a communication network N such as an in-hospital LAN via a wireless access point (AP) 20 installed in the hospital. LAN is an abbreviation for Local Area Network. The main body 1 is capable of transmitting and receiving data to and from external devices, such as an RIS 30, a PACS 40, and an analysis apparatus 50, via the communication network N. RIS is an abbreviation for Radiology Information System. PACS is an abbreviation for Picture Archiving and Communication System.

The dynamic imaging system 10 performs static imaging or dynamic imaging of a subject H by irradiating the subject H with radiation from the radiation source 2 in a state where the FPD 3 is disposed opposite the radiation source 2 with the subject H in-between. In the present embodiment, dynamic imaging refers to obtaining a series of images of the subject H by repeatedly irradiating the subject H with pulsed radiation such as X-rays at predetermined time intervals in response to a single imaging operation. Repeated irradiation in the form of pulses at predetermined time intervals is referred to as pulsed irradiation. Dynamic imaging refers to obtaining a series of images of the subject H by continuously irradiating the subject H with radiation at a low dose rate in response to one imaging operation. Continuously applying radiation without suspension is referred to as continuous irradiation. A series of images obtained by dynamic imaging is called a dynamic image. Images constituting a dynamic image may be called frames. Dynamic imaging includes moving image capturing but does not include capturing of a still image while displaying a moving image. Further, examples of a dynamic image include a moving image but do not include still images captured while displaying a moving image.

The RIS 30 generates and stores examination order information. The RIS 30 transmits the generated examination order information to the main body 1 of the dynamic imaging system 10 via the communication network N.

The PACS 40 stores and manages medical images generated by a modality, such as the dynamic imaging system 10, in association with supplementary information of the medical images. The supplementary information includes patient information and examination information. The medical image includes a still image and a dynamic image.

The analysis apparatus 50 analyzes a medical image generated by a modality such as the dynamic imaging system 10 and outputs an analysis result.

### Example of Block Configuration of Main Body 1

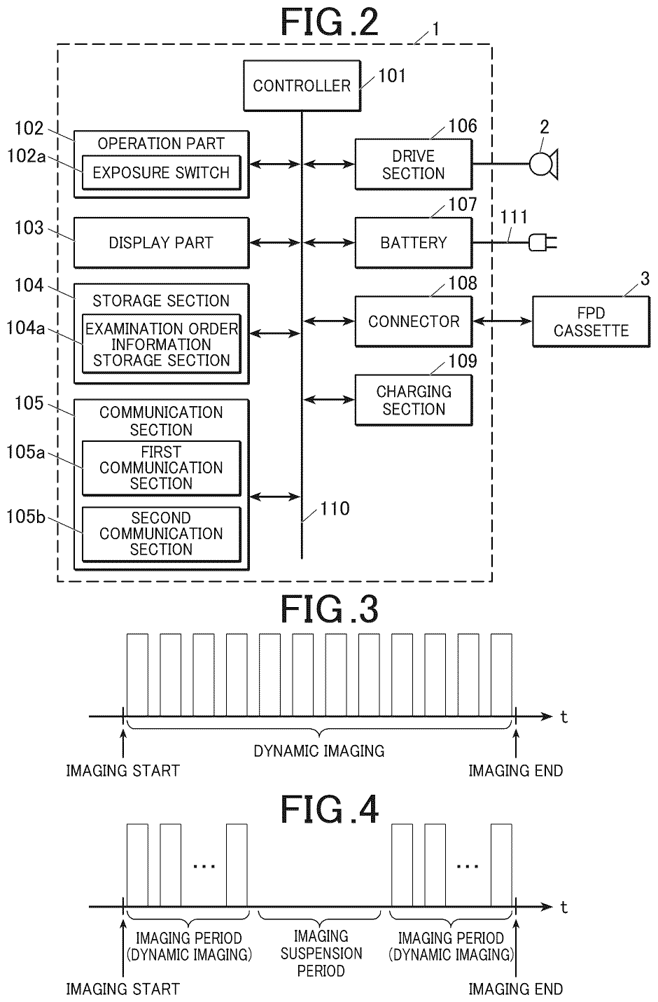

FIG. 2 is a block diagram of the main body 1 according to the first embodiment. The main body 1 includes a computer and functions as a console or an imaging control apparatus. The main body 1 includes a controller 101 (hardware processor), an operation part 102, a display part 103, a storage section 104, a communication section 105, a drive section 106, a battery 107, a connector 108, and a charging unit 109. The controller 101, the operation part 102, the display part 103, the storage section 104, the communication section 105, the drive section 106, the battery 107, the connector 108, and the charging unit 109 are connected via wires, such as a bus 110.

The controller 101 includes a processor, such as a CPU, and a memory, such as a RAM. CPU is an abbreviation for Central Processing Unit. RAM is an abbreviation for Random Access Memory. In response to an input from the operation part 102, the controller 101 reads a system program and various processing programs stored in the storage section 104, loads the program in the RAM, and executes various processes in accordance with the loaded program.

In the present embodiment, the controller 101 can execute an intermittent imaging mode as one of the imaging categories. In the intermittent imaging mode, a dynamic image or the like is captured multiple times from one time of imaging start to one time of imaging end, based on a specified imaging period and a specified imaging suspension period. The start of imaging refers to a timing at which both the FPD 3 and the radiation source 2 are ready and X-ray irradiation is permitted for the intermittent imaging order. The end of imaging refers to a timing at which the intermittent imaging order ends and dynamic imaging or the like is disabled in this order. The imaging period refers to a period from the start to the end of imaging. The imaging suspension period refers to a period in which dynamic imaging or the like is not performed during the intermittent imaging mode. Specifically, in the intermittent imaging mode, the controller 101 prioritizes displaying display-purpose images of chronologically later intermittent imaging over displaying display-purpose images of chronologically earlier intermittent imaging. For example, when new (next) intermittent imaging is started, the controller 101 stops obtaining frames of the previous intermittent imaging and obtains frames of the new (next) intermittent imaging.

The operation part 102 includes at least one of buttons, a touch screen, a touch pad, and a trackball. The operation part 102 receives a predetermined operation with a finger or a touch pen and outputs operation information corresponding to the operation to the controller 101. The operation part 102 may function as a selection part for selecting whether to prioritize displaying images of the next intermittent imaging over displaying images of the previous intermittent imaging. In this case, the user may select a priority button, or the like displayed on the screen of the display part 103 by operating the operation part 102. The operation part 102 includes an exposure switch 102a. The exposure switch 102a is a switch for the user to instruct irradiation with the radiation source 2. In the present embodiment, the user includes a medical professional, such as a doctor or a radiologist.

The display part 103 consists of a monitor, such as a liquid crystal display or an organic EL display. EL is an abbreviation for Electro Luminescence. The display part 103 displays a dynamic image or the like on a screen in accordance with an instruction of a display signal input by the controller 101. The display part 103 may function as a notification section that notifies the user that a display delay has occurred when a frame cannot be displayed within a predetermined time from a specified display timing. For example, in a case where the difference between the frame generation time by the FPD 3 and the image display time by the display part 103 is not within a predetermined time, the display part 103 notifies the user that the display delay occurs.

The storage section 104 consists of a nonvolatile semiconductor memory, a hard disk, and the like. The storage section 104 stores various programs to be executed by the controller 101, parameters required for executing processing by the programs, and data such as processing results. The storage section 104 includes an examination order information storage section 104a. The examination order information storage section 104a stores examination order information obtained from the RIS 30. The examination order information includes patient information and examination information. The patient information includes the patient ID, name, sex, age, and the hospital room (ward) of the patient to be examined. The examination information includes the examination ID, examination date, and the imaging order for each imaging to be performed in the examination. The imaging order includes an imaging region, an imaging direction, and an imaging category. The imaging category includes still image capturing and dynamic imaging, such as an intermittent imaging mode. The storage section 104 includes a temporary storage area (not shown) for temporarily storing medical image transferred from the FPD 3. Furthermore, the storage section 104 includes an image storage area (not illustrated) for storing medical images transferred from the FPD 3 for a certain period of time in association with supplementary information.

The communication section 105 includes a first communication section 105a and a second communication section 105b. The first communication section 105a performs data transmission and reception to and from the FPD 3 by wired communication or wireless communication. The second communication section 105b performs data transmission and reception to and from an external device, such as the RIS 30 or the PACS 40 connected to the communication network N via the wireless access point 20.

The drive section 106 is a circuit that drives the tube of the radiation source 2. The drive section 106 and the radiation source 2 are connected to each other via a cable.

The battery 107 supplies electric power to each part of the main body 1 and the radiation source 2. The battery 107 can be charged externally via an AC cable 111.

The connector 108 is provided inside a casing 120 and is electrically connected to the FPD 3 stored in the casing 120.

The charging section 109 charges the FPD 3 connected via the connector 108 with the power supplied by the battery 107 under the control of the controller 101.

The radiation source 2 is driven by the drive section 106 and irradiates the subject H with radiation such as X-rays. In dynamic imaging, the radiation source 2 repeatedly irradiates the subject H with pulsed radiation at predetermined time intervals, for example.

The FPD 3 is a portable radiation detector that is compatible with still imaging and dynamic imaging. The FPD 3 includes, for example, radiation detection elements arranged two-dimensionally on a glass substrate. Each of the radiation detection elements consists of a semiconductor image sensor, such as a photodiode. The radiation detection element detects radiation emitted by the radiation source 2 and transmitted through at least the subject H, based on the radiation intensity, converts the detected radiation into an electrical signal, and accumulates the electrical signal. To each radiation detection element, a switching section is connected, such as a TFT. TFT is an abbreviation for Thin Film Transistor. The switching section obtains image data by controlling accumulation and reading of the electrical signals. The FPD 3 can be either an indirect conversion type in which radiation is converted into electric signals by photoelectric conversion elements via a scintillator, or a direct conversion type in which radiation is directly converted into electric signals. In the present embodiment, the FPD 3 is a so-called self-detection type FPD. That is, the FPD 3 has an automatic detection mode function of automatically detecting irradiation. In dynamic imaging, when the FPD 3 detects the start of radiation irradiation, the imaging apparatus FPD 3 performs imaging at a set frame rate. The frames obtained by imaging are sequentially transferred to the main body 1 by a communication section (not shown). Note that the FPD 3 is not limited to the self-detection type.

Imaging may be performed using a synchronous method. In the synchronous imaging, the controller 101 controls irradiation at a frame rate set in accordance with specific timing signals, and the FPD 3 obtains frames at the frame rate set in accordance with the specific timing signals. As the synchronization method for obtaining the specific timing signals, the following method can be adopted. In a case where the FPD 3 is connected to the main body 1 via a wire, the FPD 3 may synchronize its own timer information with the timer information generated by the controller 101. Further, the FPD 3 may be controlled to maintain the synchronized state with the controller 101 even after the connection with the main body 1 is released. That is, the controller 101 and the FPD 3 synchronize with each other at the time of wired connection and continue to generate synchronization pulses and run by themselves at the time of wireless connection. The FPD 3 may perform time synchronization communication with the controller 101 over a LAN called IEEE 1588 to correct a time difference between the FPD 3 and the controller 101. For another example, the controller 101 may transmit a synchronization signal to the radiation source 2 and the FPD 3 to synchronize the radiation source 2 with the FPD 3. For another example, the controller 101 may transmit a synchronization signal to the radiation source 2 and the FPD 3 to synchronize with the radiation source 2 and the FPD 3. If a plurality of synchronization methods are available, the methods may be switched from one method to a method with higher synchronization accuracy. Note that a radiation control apparatus (not shown) may control irradiation separately from the controller 101 of the main body 1.

### Normal Dynamic Imaging Mode and Intermittent Imaging Mode

In known dynamic imaging, pulse irradiation by the radiation source 2 is continuously performed from the start to the end of imaging to acquire a dynamic image consisting of a series of frames. However, in performing imaging outside the imaging room, the total dose that can be applied from the start to the end of imaging is limited from the viewpoint of risk management. Therefore, pulse irradiation cannot be performed for a long time. Since imaging cannot be performed for a long time, images at necessary timings may not be obtained in a case where dynamic imaging is performed to check the state of a procedure of catheter insertion or to check the state inside the body immediately after a contrast medium is taken.

Therefore, the dynamic imaging system 10 can execute a normal mode and an intermittent imaging mode as dynamic imaging modes. FIG. 3 schematically illustrates pulse irradiation in the normal mode in a known art. FIG. 4 schematically illustrates pulse irradiation in the intermittent imaging mode according to the first embodiment.

In the normal mode, dynamic imaging is performed in the same way as the known art. That is, in the normal mode, pulse irradiation by the radiation source 2 is continuously performed from the start to the end of the imaging, as shown in FIG. 3. In the normal mode, one dynamic image consisting of a plurality of frames is obtained. In the intermittent imaging mode, dynamic imaging, in which pulse irradiation by the radiation source 2 is continuously performed, is intermittently performed multiple times from the start to the end of one session of imaging, based on a specified imaging period and a specified imaging suspension period, as shown in FIG. 4. In the intermittent imaging mode, multiple dynamic images, each of which consists of a series of frames, are obtained. Each execution of dynamic imaging in the intermittent imaging mode is called intermittent imaging.

In the present embodiment, in the normal mode, the radiation source 2 continues pulse irradiation from the start to the end of imaging. During the period from the start to the end of imaging in the intermittent imaging mode, the radiation source 2 performs pulse irradiation during the specified imaging period, whereas the radiation source 2 stops (suspends) pulse irradiation and stands by while rotating the anode during the specified imaging suspension period.

In the normal mode, after the imaging start is notified, the FPD 3 detects radiation and starts accumulation and reading of charges corresponding to the radiation. When the FPD 3 does not detect radiation for a predetermined period or longer, the FPD 3 determines that the imaging ends and ends the accumulation and reading of charges. In the intermittent imaging mode, after the imaging start is notified, the FPD 3 detects radiation and starts accumulation and reading of charges corresponding to the radiation. Thereafter, even if the FPD 3 does not detect radiation for a predetermined period or longer, the FPD 3 recognizes that imaging is in progress until the imaging end is notified and continues accumulation and reading of charges corresponding to the detected radiation.

### Operation Example of Controller 101 of Main Body 1

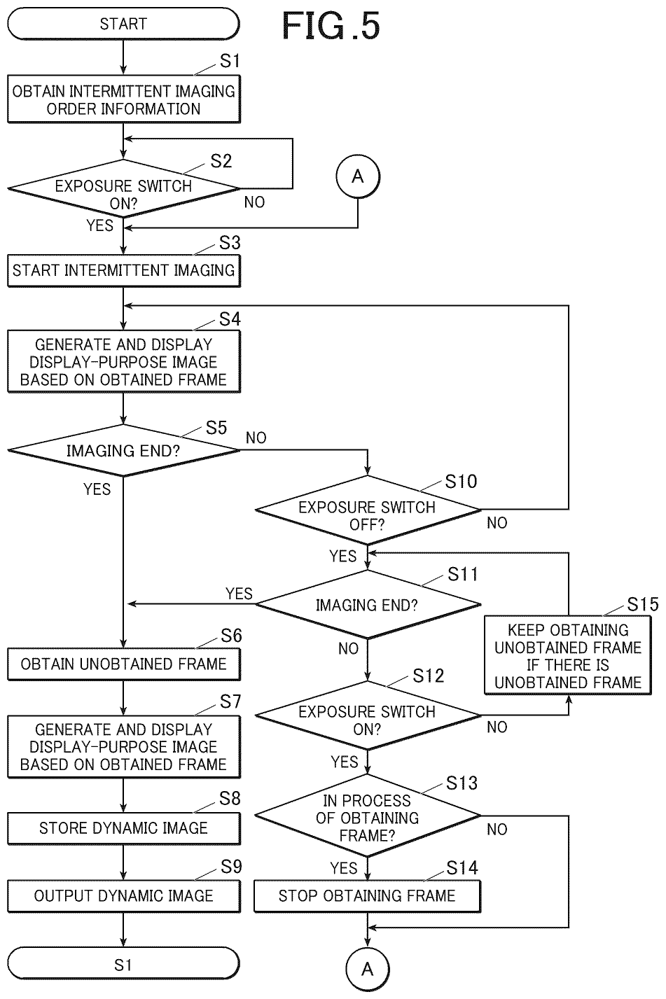

FIG. 5 is a flowchart of an operation example of the controller 101 of the main body 1 in executing the intermittent imaging mode according to the first embodiment. The controller 101 performs processes, such as the control steps illustrated in FIG. 5, by executing the programs stored in the storage section 104.

The user selects an examination of an imaging target in which dynamic imaging is to be performed from a not-illustrated examination order list screen by manipulating the operation part 102. The controller 101 of the main body 1 causes the display part 103 to display the imaging screen 130 corresponding to the selected examination.

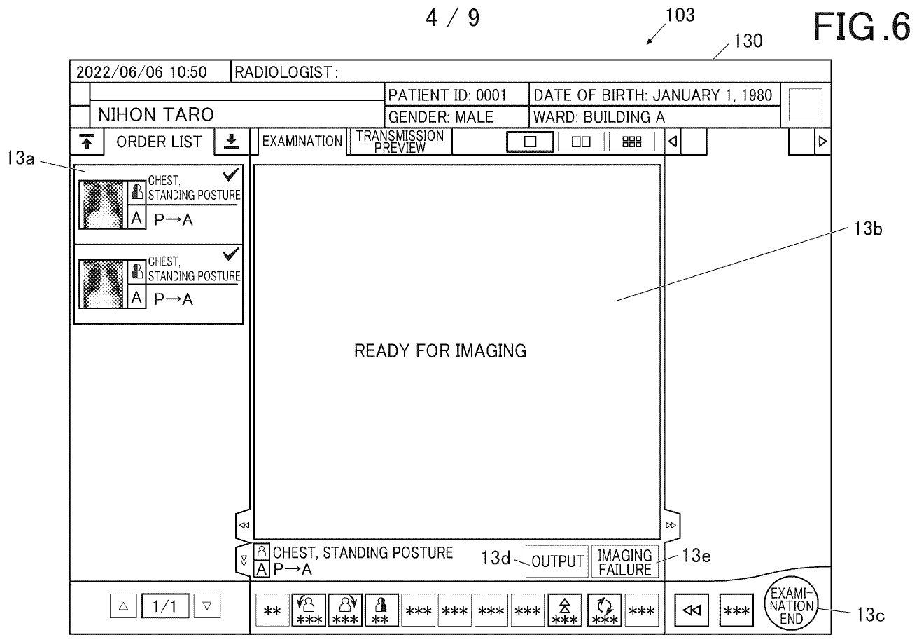

FIG. 6 illustrates an example of the imaging screen 130 displayed on the display part 103 according to the first embodiment. The imaging screen 130 includes an imaging order list 13a, an image display field 13b, an examination end button 13c, an output button 13d, and a imaging failure button 13e. The imaging order list 13a is a list of pieces of imaging order information included in the examination order information of the selected examination. The imaging order information includes an imaging region, an imaging direction, and an imaging category, such as dynamic imaging. The image display field 13b displays an image captured in the intermittent imaging mode or the like. The examination end button 13c is a button for giving an instruction to end the examination. The output button 13d is a button for instructing output of the captured image to at least one external device, such as the PACS 40 or the analysis apparatus 50. The imaging failure button 13e is a button for giving an instruction to discard an obtained image without outputting the image.

The user selects intermittent imaging order information from the imaging order list 13a on the imaging screen 130 by operating the operation part 102. The controller 101 of the main body 1 obtains the intermittent imaging order information selected by the user (step S1). The controller 101 causes the radiation source 2 and the FPD 3 to prepare for imaging. For example, the controller 101 sets irradiation conditions corresponding to the selected intermittent imaging order information to the drive section 106 and causes the drive section 106 to activate the radiation source 2 and keep the radiation source 2 in a standby state. The controller 101 transmits image reading conditions corresponding to the selected intermittent imaging order information to the FPD 3 via the first communication section 105a and causes the FPD 3 to prepare for imaging (e.g., to perform reset processing). When the reset processing ends, the FPD 3 proceeds to the automatic detection mode.

The controller 101 determines whether the exposure switch 102a has been turned on by the user (step S2). When determining that the exposure switch 102a has been turned on by the user, the controller 101 proceeds to step S3. When determining that the exposure switch 102a has not been turned on, the controller 101 continuously checks the state of the exposure switch 102a and so forth.

When the exposure switch 102a is turned on by the user, the controller 101 executes the intermittent imaging mode and starts N-th intermittent imaging (step S3). Specifically, the controller 101 causes the radiation source 2 to perform pulse irradiation by controlling the drive section 106, based on the frame rate specified in the radiation irradiation conditions. When the FPD 3 detects radiation emitted by the radiation source, the FPD 3 repeats a process of capturing a frame with an accumulation time and a reading time corresponding to the frame rate specified in the image reading conditions. The FPD 3 adds a frame number indicating an imaging order and an identification number of intermittent imaging to each of the captured frames and transmits the frames to the main body 1 by wireless communication, for example.

The controller 101 sequentially obtains a plurality of frames transmitted from the FPD 3 by wireless communication. The controller 101 generates a display-purpose image from the obtained frames and outputs the generated display-purpose image to the display part 103. The display part 103 displays the image outputted by the controller 101 on the imaging screen 130 (step S4). The image displayed on the imaging screen 130 is a dynamic image consisting of a plurality of frame images.

The controller 101 determines whether the intermittent imaging mode has ended (step S5). For example, the controller 101 may determine that the intermittent imaging mode has ended when the number of captured frames has reached a preset upper limit number of frames or when a preset imaging time has elapsed. In a case where the user presses the end button for ending the intermittent imaging mode displayed on the imaging screen 130, the controller 101 may determine that the intermittent imaging mode has ended.

If the controller 101 determines that the intermittent imaging mode has not ended, the controller 101 proceeds to step S10. The controller 101 determines whether the exposure switch 102a has been turned off by the user while executing the intermittent imaging mode (step S10). The user turns off the exposure switch 102a when ending the N-th intermittent imaging that has started in step S3.

Thereafter, when determining that the exposure switch 102a has not been turned off by the user, the controller 101 proceeds to step S4. In this case, the N-th intermittent imaging is in progress. Therefore, the controller 101 obtains frames transmitted from the FPD 3 and displays images of the obtained frames on the imaging screen 130 of the display part 103.

In step S10, when determining that the exposure switch 102a has been turned off by the user, the controller 101 proceeds to step S11. The controller 101 then determines whether the intermittent imaging mode has ended (step S11). This is because the number of captured frames may reach the upper limit number of frames or a preset imaging time may elapse when the exposure switch 102a is turned off in step S10.

When determining that the intermittent imaging mode has not ended, the controller 101 proceeds to step S12. In this case, the intermittent imaging is suspended. The controller 101 then determines whether the user has turned on the exposure switch 102a (step S12). The user turns on the exposure switch 102a again when performing the next intermittent imaging. If the controller 101 determines that the exposure switch 102a has not been turned on, the controller 101 proceeds to step S15.

If there is a frame(s) that has not yet been obtained from the FPD 3 among the frames captured by the FPD 3 in the N-th intermittent imaging period performed in step S3, the controller 101 continues to obtain the frame during the suspension period of the intermittent imaging (step S15). This is because not all the frames have been transmitted from the FPD 3 to the main body 1 during the N-th intermittent imaging period owing to a communication delay. After obtaining the unobtained frames during the suspension period of the intermittent imaging, the controller 101 returns to step S11 and determines whether the intermittent imaging mode has ended.

In step S12, when determining that the exposure switch 102a has been turned on by the user, the controller 101 proceeds to step S13. When performing the next ((N+1)th) intermittent imaging, the user turns on the exposure switch 102a.

When starting the next intermittent imaging, the controller 101 determines whether a frame(s) in the chronologically previous intermittent imaging is being obtained (step S13). That is, at the start of the next intermittent imaging, the controller 101 determines whether the first communication section 105a is in the process of receiving a frame from the FPD 3. If the controller 101 determines that a frame captured in the previous intermittent imaging is being received, the controller 101 proceeds to step S14.

The controller 101 stops obtaining the frame captured in the previous intermittent imaging (step S14). Specifically, when the first communication section 105a is in the process of receiving a frame from the FPD 3, the controller 101 causes the first communication section 105a to stop receiving the frame. Herein, the controller 101 may discard the data of the frame obtained halfway or may hold the data so that the first communication section 105a can continue receiving the data at the next acquisition. The controller 101 may cause the first communication section 105a to obtain all the frames transmitted from the FPD 3 and then discard the frames. At the start of the next intermittent imaging, the controller 101 may send to the FPD 3 an instruction to stop transmitting the frames captured in the previous intermittent imaging. After stopping the acquisition of frames captured in the previous intermittent imaging, the controller 101 proceeds to step S3.

In Step S13, when determining that a frame(s) captured in the chronologically previous intermittent imaging is not being obtained, the controller 101 proceeds to step S3. This is the case where the first communication section 105a of the main body 1 has received, from the FPD 3, all the frames captured in the previous intermittent imaging at the start of the next intermittent imaging.

If the exposure switch 102a is turned on by the user, the controller 101 performs the next new intermittent imaging (step S3). The controller 101 generates a display-purpose image based on a plurality of frames transmitted from the FPD 3 in the next intermittent imaging and outputs the generated image to the display part 103. The display part 103 displays the image output by the controller 101 on the imaging screen 130 (step S4).

Thereafter, when determining that the intermittent imaging mode has ended (step S5), the controller 101 proceeds to step S6. Similarly, when determining in step S11 that the intermittent imaging mode has ended, the controller 101 proceeds to step S6. These are the cases where the last intermittent imaging among the multiple times of intermittent imaging set in the intermittent imaging mode has been completed. At the end of the last intermittent imaging, the controller 101 obtains a frame(s) that has not been obtained yet among the plurality of frames in the last intermittent imaging (step S6).

The controller 101 generates display-purpose images from the obtained multiple frames and outputs the generated multiple images to the display part 103. The display part 103 displays the dynamic image consisting of the multiple images output by the controller 101 on the imaging screen 130 (step S7).

The controller 101 attaches supplementary information to the dynamic image including frames obtained in the respective times of intermittent imaging and stores the dynamic image having the supplementary information in the image storage area of the storage section 104 (step S8).

The controller 101 transmits the generated dynamic image to at least one of the PACS 40 and the analysis apparatus 50 via the second communication section 105b (step S9). By the above series of processing, the intermittent imaging mode is performed.

The following process can also be added to the flowchart in FIG. 5, which illustrates the flow of the intermittent imaging mode. The controller 101 of the main body 1 preferably performs at least one of offset correction, gain correction, defect correction, afterimage correction, and gradation correction before displaying a frame on the display part 103. When performing offset correction, the controller 101 may obtain an image for offset correction (dark image) at the time of the first intermittent imaging and perform correction using the same offset correction image during the imaging period. Thus, in offset correction, internal processing can be simplified and accelerated. The controller 101 may obtain an offset correction image for each intermittent imaging in the intermittent imaging mode.

The controller 101 may change the correction processing, the gradation processing, and so forth between display-purpose images and recording-purpose images. Herein, the controller 101 may make the pixel pitch of the display-purpose images larger than that of the recording-purpose images. Since the data size of the display-purpose images can be reduced, the display speed can be improved.

Frame information may be overlaid on an image. The frame information indicates which time of dynamic imaging a frame belongs to or indicates what the frame number is and to which time of dynamic imaging a frame belongs to in the intermittent imaging mode. Instead of being overlaid on the image, the frame information may be added to supplementary information of the image or to header information. The image data may include information that identifies which time of intermittent imaging the frame belongs to. This allows displaying a first image or segmented display in units of intermittent imaging. To calculate the delay time of the frame display, information indicating the start time of the corresponding intermittent imaging may be added to the frame. In a case where multiple FPDs 3 perform imaging simultaneously and the captured images are combined to form wide images, frames corresponding to the same irradiation timing may be used to generate a display-purpose image.

### Flow of Intermittent Imaging Mode

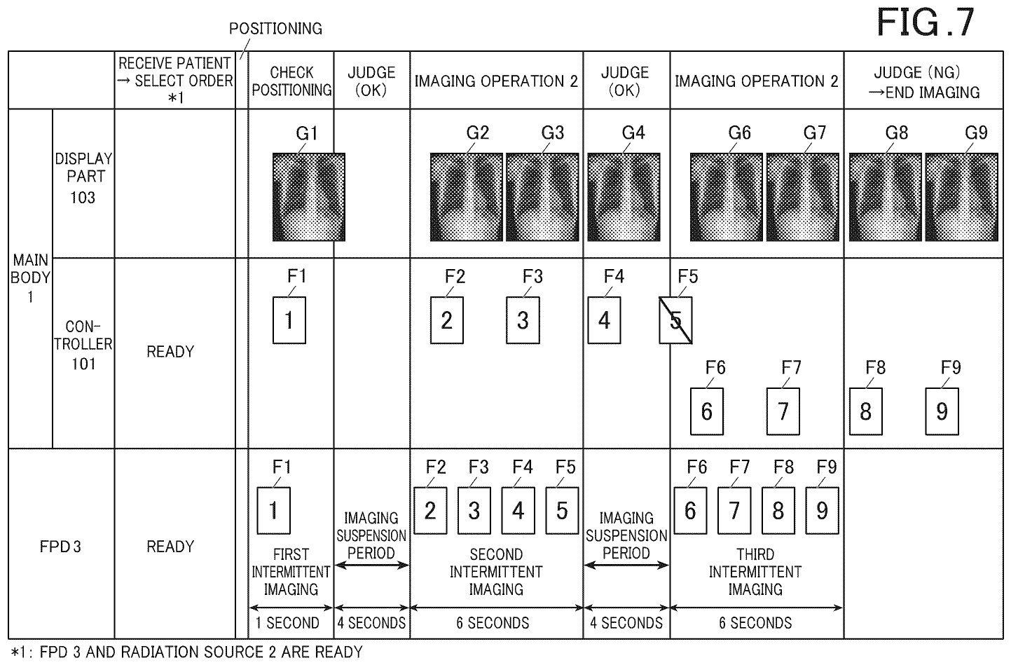

FIG. 7 illustrates a flow of intermittent imaging by the FPD 3, frame acquisition by the controller 101 of the main body 1, and image display by the display part 103 of the main body 1 when the intermittent imaging mode is executed according to the present embodiment. In the present embodiment, the method of displaying all the frame images obtained by the main body 1 in each intermittent imaging as illustrated in FIG. 7 is called a first display mode. To facilitate understanding of the invention, FIG. 7 illustrates a small number of frames captured in the intermittent imaging mode. Further, the time of each phase in FIG. 7 is an example and is not limited to the time shown in FIG. 7.

First, a terminal installed in a radiology department or the like receives a patient. Examination information including order information of the patient is transmitted from the RIS or the like to the main body 1. When the user selects an intermittent imaging order on the imaging screen 130, the controller 101 of the main body 1 sets imaging conditions for the radiation source 2, the FPD 3, and so forth, based on the selected intermittent imaging order. The user, such as a radiologist, guides the patient to a predetermined position, depending on the region to be imaged.

When the exposure switch 102a is turned on by the user, the controller 101 performs the first intermittent imaging. The first intermittent imaging is for checking the position of the patient. Therefore, a still image of one frame is captured in the first intermittent imaging. The radiation source 2 irradiates the subject H with radiation based on the specified irradiation conditions. The FPD 3 detects the radiation emitted by the radiation source 2 and captures a frame F1 for checking the position, based on the specified reading conditions. The FPD 3 wirelessly transmits the captured frame F1 to the main body 1. The first intermittent imaging for checking the position may be omitted.

The controller 101 of the main body 1 obtains the frame F1 during the period of the first intermittent imaging. The controller 101 generates an image G1 of the obtained frame F1 and outputs the generated image G1 to the display part 103. The display part 103 displays the image G1 for checking the position outputted by the controller 101 on the imaging screen 130. The user checks the image G1 on the imaging screen 130 to determine whether the patient is correctly positioned.

When the patient is correctly positioned, after a certain imaging suspension period, the user turns on the exposure switch 102a to perform the second intermittent imaging. The certain imaging suspension period is a period between when the exposure switch 102a is turned off at the end of the first intermittent imaging and when the exposure switch 102a is turned on at an uncertain timing at the start of the second intermittent imaging. The controller 101 performs the second intermittent imaging in response to the exposure switch 102a being turned on. The second intermittent imaging is for obtaining a dynamic image for diagnosis. The radiation source 2 irradiates the subject H with radiation based on irradiation conditions, such as a specified frame rate. The FPD 3 detects the radiation emitted by the radiation source 2 and captures frames F2 to F5 for diagnosis, based on the specified reading conditions. The FPD 3 wirelessly transmits the captures frames F2 to F5 to the main body 1.

The first communication section 105a of the main body 1 wirelessly receives the frame F2 and so forth from the FPD 3. However, owing to a delay in wireless communication, the first communication section 105a cannot receive, within the second intermittent imaging period, all the frames F2 to F5 captured in the second intermittent imaging period. The controller 101 of the main body 1 obtains the frames F2 and F3 from the FPD 3 within the second intermittent imaging period. The controller 101 obtains the frame F4, which could not be obtained within the second intermittent imaging period, from the FPD 3 in the imaging suspension period after the second intermittent imaging period ends.

The controller 101 generates display-purpose images G2 to G4 from the obtained frames F2 to F4 and outputs the generated images G2 to G4 to the display part 103. The display part 103 displays the images G2 to G4 output by the controller 101 on the imaging screen 130. The image G2 to G4 form a dynamic image for diagnosis and include information such as the continuous motion of the imaged region of the subject. The user performs diagnosis of the imaged region of the patient while checking the images G2 to G4 on the imaging screen 130. The user may also determine whether the images G2 to G4 include an imaging failure.

After checking the images G2 to G4 and after a certain imaging suspension period, the user turns on the exposure switch 102a to perform the third intermittent imaging. The certain imaging suspension period is a period between when the exposure switch 102a is turned off at the end of the second intermittent imaging and when the exposure switch 102a is turned on at an uncertain timing at the start of the third intermittent imaging. The controller 101 performs the third intermittent imaging in response to the exposure switch 102a being turned on. The third intermittent imaging is for obtaining a dynamic image for diagnosis. The radiation source 2 irradiates the subject H with radiation based on irradiation conditions, such as a specified frame rate. The FPD 3 detects the radiation emitted by the radiation source 2 and captures frames F6 to F9 for diagnosis, based on the specified reading conditions. The FPD 3 wirelessly transmits the captured frames F6 to F9 to the main body 1.

In the present embodiment, at the start of the third intermittent imaging, the remaining frame F5 captured in the second intermittent imaging period is in the process of being transmitted from the FPD 3 to the main body 1. That is, at the start of the third intermittent imaging, the controller 101 of the body 1 is in the process of obtaining the frame F5 captured in the previous second intermittent imaging. In this case, at the start of the third intermittent imaging, the controller 101 stops obtaining the frame F5 captured in the second intermittent imaging and gives priority to obtaining a frame F6 and so forth that is newly captured in the third intermittent imaging. Specifically, the controller 101 of the main body 1 obtains the frames F6 and F7 from the FPD 3 during the third intermittent imaging period. The controller 101 obtains the frames F8 and F9, which could not be obtained within the third intermittent imaging period, from the FPD 3 in the imaging suspension period after the third intermittent imaging period ends.

The controller 101 generates display-purpose images G6 to G9 based on the obtained frames F6 to F9 and outputs the generated images G6 to G9 to the display part 103. The display part 103 sequentially displays the images G6 to G9 output by the controller 101 on the imaging screen 130. The images G6 to G9 form a dynamic image for diagnosis and include information such as the continuous motion of the imaged region of the subject. The user performs diagnosis of the imaged region of the patient while checking the images G6 to G9 on the imaging screen 130. The user may also determine whether the images G6 to G9 include an imaging failure.

### First Modification Example

In the above-described example, the first display mode is adopted in which all the frame images obtained by the main body 1 in each intermittent imaging are displayed. However, the display method is not limited thereto. For example, only the image of the first frame (front image) among all the frames obtained by the main body 1 in each intermittent imaging may be displayed on the display part 103. The display method of displaying only the image of the first frame among the frames in each intermittent imaging in the first modification example is called a second display mode.

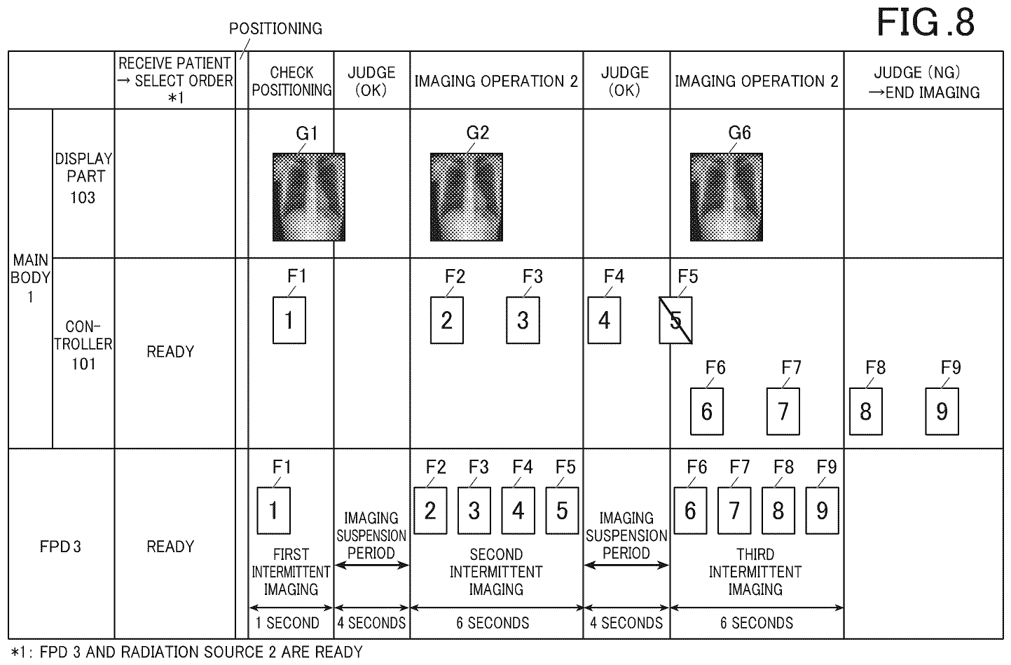

FIG. 8 illustrates a flow of intermittent imaging by the FPD 3, frame acquisition by the controller 101 of the main body 1, and image display by the display part 103 of the main body 1 when the intermittent imaging mode is executed according to the first modification example. To facilitate understanding of the invention, FIG. 8 illustrates a small number of frames captured in the intermittent imaging mode. Further, the time of each phase in FIG. 8 is an example and is not limited to the time shown in FIG. 8.

When the position of the patient is checked and determined to be appropriate in the first intermittent imaging, the second intermittent imaging is performed. The FPD 3 sequentially captures the frames F2 to F5 in the second intermittent imaging period. The controller 101 of the main body 1 sequentially obtains the frames F2 and F3 from FPD 3 in the second intermittent imaging period. The controller 101 obtains the frame F4, which could not be obtained within the second intermittent imaging period owing to a delay in communication, from the FPD 3 in the imaging suspension period after the second intermittent imaging period. In this case, the controller 101 generates only the image G2 of the first frame F2 among the frames F2 to F4 obtained from the FPD 3 and outputs the generated image G2 to the display part 103. The display part 103 displays the image G2 output by the controller 101 on the imaging screen 130.

When the image G2 is checked in the second intermittent imaging, the third intermittent imaging is performed. At the start of the third intermittent imaging, the controller 101 of the main body 1 is in the process of obtaining the frame F5 from the FPD 3 that could not be obtained during the second intermittent imaging period owing to a communication delay. In this case, at the start of the third intermittent imaging, the controller 101 stops obtaining the frame F5 captured in the second intermittent imaging and gives priority to obtaining the frame F6 and so forth that is captured in the third intermittent imaging.

The FPD 3 sequentially captures the frames F6 to F9 in the third intermittent imaging period. The controller 101 of the main body 1 sequentially obtains the frames F6 and F7 from the FPD 3 during the third intermittent imaging period. The controller 101 of the main body sequentially obtains the frames F8 and F9, which could not be obtained during the third intermittent imaging period due to the communication delay, from the FPD 3 in a period after the third intermittent imaging. In this case, the controller 101 generates only the image G6 of the first frame F6 among the frames F6 to F9 obtained from the FPD 3 and outputs the generated image G6 to the display part 103. The display part 103 displays the image G6 output by the controller 101 on the imaging screen 130.

In the above-described first modification example, the specific frame to be displayed with highest priority is the first frame. However, the specific frame may be another frame. For example, the specific frame may be an N-th frame. N is a natural number. When the total number of frames is less than N, the specific frame may be the last frame. Herein, N is a natural number. Another specific frame may be a frame including a region of interest or a frame having a signal value equal to or greater than a predetermined value. Whether the signal value of the frame is equal to or greater than the predetermined value may be determined using, for example, any of an average value, a median value, a mode value, and histogram analysis of signal values in the frame. Whether the signal value of the frame is equal to or greater than the predetermined value may be determined by a difference from the last frame in the previous intermittent imaging. In the first modification example, frames other than the first frame of each intermittent imaging to be displayed by the main body 1 are obtained as well as the first frame. However, frames other than the first frame to be displayed may not be obtained.

### Second Modification Example

The method of displaying images on the display part 103 of the main body 1 may be other than the first display mode and the second display mode described above. In the second modification example, images of half of all the frames obtained by the main body 1 during each intermittent imaging may be displayed on the display part 103, for example. That is, the main body 1 obtains frames at a frame rate lower than the imaging frame rate of the FPD 3 and displays images of the obtained frames. The display method of displaying images at a frame rate lower than the imaging frame rate in the second modification example is referred to as a third display mode.

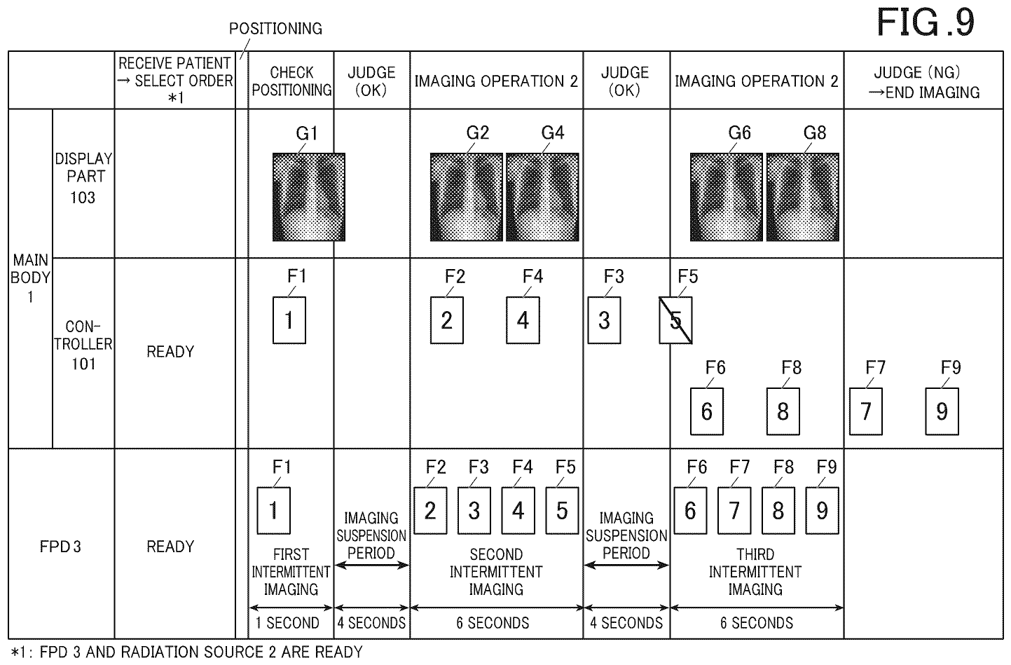

FIG. 9 illustrates a flow of intermittent imaging by the FPD 3, frame acquisition by the controller 101 of the main body 1, and image display by the display part 103 of the main body 1 when the intermittent imaging mode is executed according to the second modification example. To facilitate understanding of the invention, FIG. 9 illustrates a small number of frames as frames captured in the intermittent imaging mode. Further, the time of each phase in FIG. 9 is an example and is not limited to the time shown in FIG. 9.

When the position of the patient is checked and determined to be correct in the first intermittent imaging, the second intermittent imaging is performed. The FPD 3 sequentially captures the frames F2 to F5 in the second intermittent imaging period. In the second intermittent imaging period, the controller 101 of the main body 1 sequentially obtains the frames F2 and F4 at a half frame rate lower than the imaging frame rate. The controller 101 obtains the frame F3, which was not obtained within the second intermittent imaging period owing to a communication delay, from the FPD 3 in the imaging suspension period after the second intermittent imaging period. Among the frames F2 to F4 obtained from the FPD 3, the controller 101 generates the images G2 and G4 of the frames F2 and F4. The display part 103 displays the images G2 and G4 generated by the controller 101 on the imaging screen 130.

When the image G2 is checked in the second intermittent imaging, the third intermittent imaging is performed. At the start of the third intermittent imaging, the controller 101 of the main body 1 is in the process of obtaining the frame F5 from the FPD 3 that could not be obtained within the second intermittent imaging period owing to a communication delay. In this case, at the start of the third intermittent imaging, the controller 101 stops obtaining the frame F5 of the second intermittent imaging and gives priority to obtaining the frame F6 and so forth that is captured in the third intermittent imaging.

The FPD 3 sequentially captures the frames F6 to F9 in the third intermittent imaging period. In the third intermittent imaging period, the controller 101 of the main body 1 sequentially obtains the frames F6 and F8 at a half frame rate lower than the imaging frame rate. The controller 101 of the main body sequentially obtains the frames F7 and F9, which could not be obtained within the third intermittent imaging period due to the communication delay, from the FPD 3 in a period after the third intermittent imaging. The controller 101 obtains the frame F8, which could not be obtained within the third intermittent imaging period owing to a communication delay, from the FPD 3 in the imaging suspension period after the third intermittent imaging period. Among the frames F6 to F9 obtained from the FPD 3, the controller 101 generates only the images G6 and G8 of the frames F6 and F8. The display part 103 displays the images G6 and G8 generated by the controller 101 on the imaging screen 130.

According to the present embodiment, the first modification example, and the second modification example, if the main body 1 is in the process of obtaining a frame captured in the previous intermittent imaging at the start of the new (next) intermittent imaging, the controller 101 of the body 1 stops obtaining the frame captured in the previous intermittent imaging and gives priority to obtaining a frame of the next intermittent imaging. Thus, display of images captured in the new intermittent imaging is prioritized over display of images captured in the previous intermittent imaging, so that images can be displayed at necessary timing for the user. This allows the user to certainly check the state of the subject in real time during the new intermittent imaging, such as the state of catheter insertion or changes in the state of the inside of the body immediately after the patient takes the contrast agent. Further, according to the first embodiment, obtainment of a frame in the previous intermittent imaging is stopped. This secures a communication band and reduce the load of obtaining a frame.

### Second Embodiment

In a case where a series of frames intermittently captured by the FPD 3 are transmitted to the main body 1 by wireless communication, the user cannot determine whether images are displayed on the display part 103 in real time or with a delay. Therefore, when a display delay occurs, the user may not notice the display delay. In the second embodiment, when a delay in displaying images occurs, the user is notified that the display delay has occurred and/or that a communication standard or the like does not meet imaging conditions, for example. In the following description, components substantially common to those of the first embodiment are denoted by the same reference numerals, and common description will be omitted or simplified.

### First Notification Means

First, a first notification means is described. The first notification means notifies the user of a display delay, based on a difference between the frame generation time and the display time. The controller 101 of the main body 1 calculates the time at which the frame was generated (frame generation time), based on the frame number appended to the frame obtained from the FPD 3 and the time at which the intermittent imaging of the frame has started. The intermittent imaging start time may be obtained from the controller 101 or from a radiation control apparatus (not illustrated). When displaying the image of the obtained frame on the imaging screen 130, the controller 101 determines whether the difference between the time at which the frame is displayed and the frame generation time is within a predetermined time.

When the difference is within the predetermined time, the controller 101 determines that the display delay is within an allowable range. In this case, the controller 101 performs normal image display without notifying the user of the display delay. On the other hand, when the difference exceeds the predetermined time, the controller 101 determines that the display delay exceeds the allowable range. In this case, the controller 101 performs normal image display and notifies the user that the display delay has occurred. For example, the controller 101 may delete the image with the display delay in the dynamic image obtained by the intermittent imaging or may not display the image with the display delay. Among the images constituting the dynamic image obtained in the intermittent imaging, the controller 101 may change the image quality of the image with the display delay to a lower quality and display the image. For another example, the controller 101 may blur the image with the display delay and display the blurred image. The controller 101 may attach information, such as a delay mark, to the image with the display delay, in displaying the image. Further, the controller 101 may display information on whether a display delay has occurred on the imaging screen 130, based on the calculated difference, or may display the calculated difference on the imaging screen 130. The controller 101 may notify the user of the display delay by voice, warning sound, or vibration, for example.

### Second Notification Means

Next, a second notification means is described. The second notification means notifies the user of a display delay, based on the frame obtaining speed or the like. The controller 101 of the main body 1 calculates the time at which the frame was generated (frame generation time), based on the frame number appended to the frame obtained from the FPD 3 and the time at which the intermittent imaging of the frame has started. The intermittent imaging start time may be obtained from the controller 101 or from a radiation control apparatus (not illustrated). The controller 101 calculates the frame obtaining speed, based on the transfer time between the time at which the frame was generated by the FPD 3 and the time at which the frame was obtained by the main body 1, and the data size of the frame.

When the frame obtaining speed of frames to be displayed on the imaging screen 130 is less than a reference speed, the controller 101 notifies the user that the frame obtaining speed is not satisfied. Specifically, when the calculated obtaining speed is lower than the reference speed, the controller 101 determines that the imaging conditions are not satisfied. If the calculated obtaining speed is lower than the reference speed, the controller 101 may display the current communication speed and an expected communication speed on the imaging screen 130. Further, for example, when a link speed of Wi-Fi (Registered trademark) (i.e., a communication standard) is less than a specified value, the controller 101 may determine that the imaging conditions are not satisfied. When the communication standard is less than the specified value, the controller 101 may display the current communication standard and the expected communication standard on the imaging screen 130. The notification that the obtaining speed is lower than the reference speed and the notification that the communication standard is lower than the reference value may be displayed before the intermittent imaging mode is performed or during the intermittent imaging mode is performed.

When the communication environment does not satisfy the conditions, the controller 101 may change the communication condition. Since changing the communication conditions during the intermittent imaging mode may cause a communication failure, it is preferable that the communication conditions be changed before the intermittent imaging mode is performed. Specifically, when the received signal strength indicator (RSSI) of the wireless communication is low, the controller 101 may decrease the maximum data rate to suppress the retry. If a lot of communication noise occurs, the controller 101 may change wireless channels.

### Examples of Displaying First Notification Means and Second Notification Means

Specific examples of the first notification means and the second notification means are described. The examples before the intermittent imaging mode and during the intermittent imaging mode are described separately.

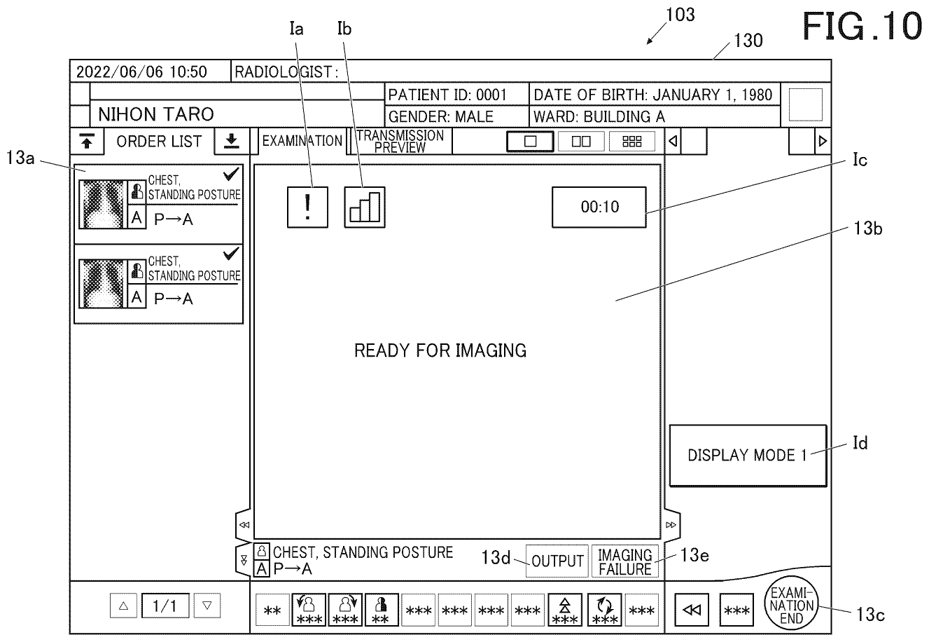

FIG. 10 illustrates an example of various kinds of information displayed on the imaging screen 130 of the display part 103 before the intermittent imaging mode is executed according to the second embodiment. In the image display field 13b of the imaging screen 130, first warning information Ia is displayed. The first warning information Ia notifies the user that a communication environment (e.g., the wireless radio field intensity) does not satisfy the imaging conditions. The first warning information Ia is, for example, an exclamation mark. The first warning information Ia may be displayed when the obtaining speed of images is less than a specified value. The image obtaining speed can be calculated by performing dummy communications of frames between the FPD 3 and the main body 1 before the intermittent imaging mode starts. Further, the first warning information Ia may be displayed when the communication standard at the time of obtaining images is less than a specified value. The communication standard at the time of obtaining images can be obtained from a network interface, such as from an OS or a driver by an API, or example.

The second warning information Ib for notifying the user of the deviation level of the communication environment from an expected value is displayed in the image display field 13b of the imaging screen 130. The second warning information Ib is, for example, an icon that shows the strength level of the radio wave intensity by the number of bars. Specifically, when the radio wave intensity is good, three bars are displayed; and when the radio wave intensity is bad, one bar is displayed. The second warning information Ib may be displayed when the communication speed for obtaining images is less than a specified value.

In the image display field 13b of the imaging screen 130, available imaging time information Ic is displayed. The available imaging time information Ic indicates a time during which imaging can be performed in the intermittent imaging mode. As the available image time information Ic, a minimum value is used that takes into account of at least one of a specified value, the remaining battery of the battery 107, and time during which synchronization can be maintained if imaging is performed in a synchronous manner. For example, when the available imaging time is 10 seconds, a numeric value of “00:10” is displayed in the image display field 13b as the available imaging time information Ic.

In a region adjacent to the image display field 13b of the imaging screen 130, currently selected display mode information Id is displayed. The display mode is, for example, any of the first display mode, the second display mode, and the third display mode described above. For example, when the first display mode is selected as the display method for the intermittent imaging mode, the words of “display mode 1” are displayed as the display mode information Id in the region adjacent to the image display field 13b. The position where the display mode information Id is displayed is not limited to the region adjacent to the image display field 13b but may be the image display field 13b, for example.

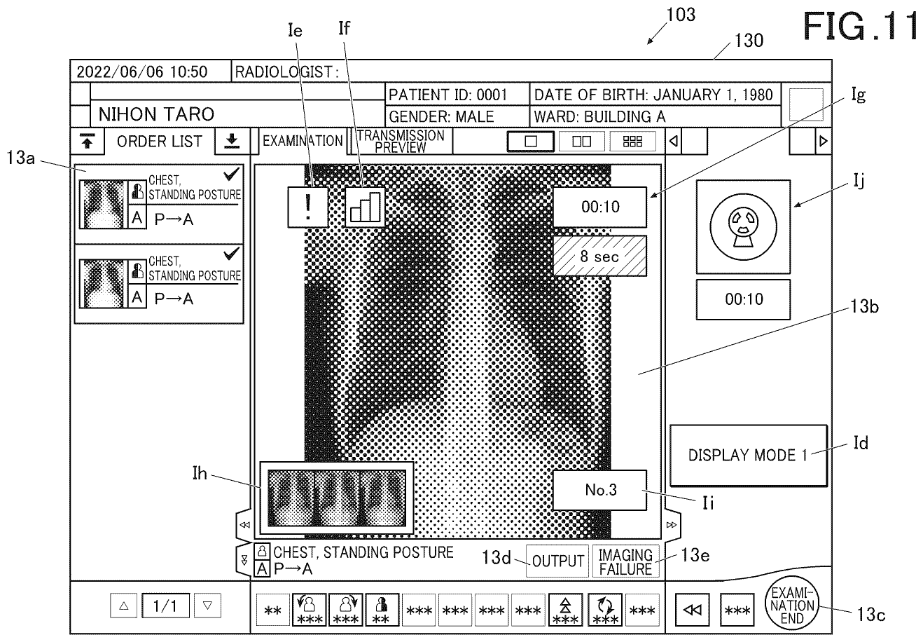

FIG. 11 illustrates an example of various kinds of information displayed on the imaging screen 130 of the display part 103 while the intermittent imaging mode is executed according to the second embodiment. Third warning information Ie for notifying the user that the difference between the image display time and the image generation time is equal to or greater than a specified value is displayed in the image display field 13b of the imaging screen 130. That is, the third warning information Ie notifies the user that an image display delay has occurred. The third warning information Ie is, for example, an exclamation mark. The third warning information Ie may be displayed when the communication speed at the time of obtaining images is lower than a specified value.

Fourth warning information If for notifying the user that the difference between the image display time and the image generation time is equal to or greater than a specified value is displayed in the image display field 13b of the imaging screen 130. The fourth warning information If is an icon that shows the difference level between the image display time and the image generation time by the number of bars. For example, if the difference indicating the display delay is large, one bar is displayed. If the difference indicating the display delay is small, three bars are displayed. The fourth warning information If may be displayed when the communication speed at the time of obtaining images is lower than a specified value.

In the image display field 13b of the imaging screen 130, time information Ig indicating an elapsed imaging time or a remaining imaging time in the intermittent imaging mode is displayed. The remaining imaging time available for imaging is calculated in consideration of at least either the remaining battery of the battery 107 or time during which synchronization can be maintained if imaging is performed by a synchronous method, for example. The elapsed imaging time includes the total imaging time in the entire intermittent imaging mode and the elapsed time in the current intermittent imaging. The total imaging time and the elapsed time may be displayed in a different manner. For example, as the total imaging time, a numerical value of “01:10” is displayed; and as the elapsed time in the current intermittent imaging, a bar indicating the ratio of the elapsed time to the intermittent imaging time is displayed, in the image display field 13b. Specifically, a linear or circular progress bar is displayed with the upper limit of the intermittent imaging time set to 100%. In this case, the elapsed time or the remaining time of the current intermittent imaging may be displayed next to the progress bar. For example, when intermittent imaging is performed for eight seconds, a numerical value of “8 sec” is displayed inside the bar.

In the image display field 13b of the imaging screen 130, past image information Ih is displayed. The past image information Ih indicates, as indexes, images obtained in the past intermittent imaging in the intermittent imaging mode. The past image information Ih is an image(s) representing a plurality of images obtained in the past intermittent imaging and consists of one or more images.

In the image display field 13b of the imaging screen 130, number information Ii is displayed. The number information Ii indicates specific intermittent imaging among multiple times of intermittent imaging constituting the intermittent imaging mode. For example, when the third intermittent imaging is in progress, “No. 3” is displayed as the number information Ii in the image display field 13b of the imaging screen 130. When an upper limit is set for the number of times of intermittent imaging, the upper limit for the number of times of imaging may be displayed next to the number of the current intermittent imaging on the imaging screen 130. For example, if the upper limit of the intermittent imaging is five times and currently the third intermittent imaging is in progress, “3/5” is displayed in the image display field 13b of the imaging screen 130.

In the field adjacent to the image display field 13b of the imaging screen 130, preparation information Ij is displayed during the imaging suspension period of the intermittent imaging mode. The preparation information Ij indicates the imaging preparation state for the next intermittent imaging and a waiting time in the next intermittent imaging. For example, when the next intermittent imaging is being prepared, an icon indicating that imaging is being prepared and a time until the preparation for the next intermittent imaging is completed are displayed as the preparation information Ij in the area adjacent to the image display field 13b. The display position of the preparatory information Ij is not limited to the area adjacent to the image display field 13b but may be the image display field 13b, for example.

According to the second embodiment, various kinds of information, such as the first warning information Ia, are displayed on the imaging screen 130 when an image display delay occurs and when the communication standard does not satisfy the imaging conditions. This allows the user to view various kinds of information displayed on the imaging screen 130 and recognize X-ray irradiation, a display delay of images with respect to the frame generation time, and so forth before or during the intermittent imaging mode. Further, the user can check the warning regarding the communication standard to recognize that the communication environment of the imaging site does not satisfy the imaging conditions or that an abnormality occurs in the communication standard.

Although the preferred embodiment of the present disclosure has been described in detail with reference to the accompanying drawings, the technical scope of the present disclosure is not limited to such examples. Further, those to which various modification examples and improvements have been applied naturally belong to the technical scope of the present disclosure within the category of the technical idea described in the scope of the claims of those skilled in the art.

1. A radiographic imaging control apparatus that comprises a hardware processor and outputs a display-purpose image generated based on a series of frames, the series of frames being captured by moving image capturing of a subject with radiation, wherein
when the moving image capturing is intermittently performed multiple times from one time of imaging start to one time of imaging end, the hardware processor prioritizes displaying a display-purpose image of second moving image capturing over displaying a display-purpose image of first moving image capturing, the second moving image capturing being performed later than the first moving image capturing.

2. The radiographic imaging control apparatus according to claim 1, wherein when the second moving image capturing starts, the hardware processor outputs the display-purpose image of the second moving image capturing to a display.

3. The radiographic imaging control apparatus according to claim 1, wherein the hardware processor displays a display-purpose image of a specific frame among the frames captured by the moving image capturing.

4. The radiographic imaging control apparatus according to claim 3, wherein the specific frame is a first frame.

5. The radiographic imaging control apparatus according to claim 3, wherein the specific frame is a frame obtained at a rate lower than a frame rate of the moving image capturing.

6. The radiographic imaging control apparatus according to claim 3, wherein:
the specific frame is an N-th frame, or
the specific frame is a last frame when a total number of frames is less than N,
where N is a natural number.

7. The radiographic imaging control apparatus according to claim 3, wherein the specific frame has a signal value greater than or equal to a predetermined value.

8. The radiographic imaging control apparatus according to claim 3, wherein the specific frame includes a region of interest.

9. The radiographic imaging control apparatus according to claim 1, wherein when the second moving image capturing starts, the hardware processor stops obtaining a frame of the first moving image capturing.

10. The radiographic imaging control apparatus according to claim 1, wherein, after the second moving image capturing ends, the hardware processor obtains a frame that has not been obtained yet among frames captured by the second moving image capturing.

11. The radiographic imaging control apparatus according to claim 1, further comprising a selection unit that receives selection on whether to prioritize displaying the display-purpose image of the second moving image capturing over displaying the display-purpose image of the first moving image capturing.

12. The radiographic imaging control apparatus according to claim 3, further comprising a notification unit, wherein when a display-purpose image of a frame among the frames cannot be displayed within a predetermined time from a specific display timing of the frame, the notification unit notifies a user that a display delay is occurring.

13. The radiographic imaging control apparatus according to claim 12, wherein:
the hardware processor lowers image quality of the display-purpose image of the frame having the display delay, or
the hardware processor does not display the display-purpose image of the frame having the display delay.

14. A radiographic imaging control method for a radiographic imaging control apparatus that outputs a display-purpose image generated based on a series of frames, the series of frames being captured by moving image capturing of a subject with radiation, wherein
when the moving image capturing is intermittently performed multiple times from one time of imaging start to one time of imaging end, displaying a display-purpose image of second moving image capturing is prioritized over displaying a display-purpose image of first moving image capturing, the second moving image capturing being performed later than the first moving image capturing.

15. A non-transitory computer-readable storage medium storing a program that causes a computer of a radiographic imaging control apparatus that outputs a display-purpose image generated based on a series of frames, the series of frames being captured by moving image capturing of a subject with radiation, wherein
when the moving image capturing is intermittently performed multiple times from one time of imaging start to one time of imaging end, the program causes the computer to prioritize displaying a display-purpose image of second moving image capturing over displaying a display-purpose image of first moving image capturing, the second moving image capturing being performed later than the first moving image capturing.
